(19) United States
(12) Patent Application Publication
(10) Pub. No.: US 2010/0016704 A1
(43) Pub. Date: Jan. 21, 2010

(54) METHOD AND SYSTEM FOR MONITORING A CONDITION OF AN EYE

(76) Inventors: John F. Naber, Goshen, KY (US); Douglas J. Jackson, New Albany, IN (US); Henry J. Kaplan, Louisville, KY (US); Julia Aebersold, Floyds Knobs, IN (US); Kevin Walsh, Louisville, KY (US); Robert S. Keynton, Louisville, KY (US); Thomas Roussel, Louisville, KY (US); Paul van der Pol, Louisville, KY (US)

Correspondence Address: FOLEY & LARDNER LLP, 150 EAST GILMAN STREET, P.O. BOX 1497, MADISON, WI, 53701-1497, US

(21) Appl. No.: 12/174,458

(22) Filed: Jul. 16, 2008

(51) Int. Cl. A61B 3/16

(52) U.S. Cl. 600/398

(57) ABSTRACT

A method, system, and computer-readable medium are provided which monitor a condition of an eye. The system includes a communication interface and a processor operably coupled to the communication interface. The communication interface is configured to receive an intraocular pressure measurement datum and a time datum associated with the time the intraocular pressure measurement was measured using an eye measurement system. The processor is configured to receive the intraocular pressure measurement datum and the time datum, to receive a dispensed amount datum associated with an amount of a drug administered to an eye of a user and a second time datum associated with the time the drug was administered, and to store the received intraocular pressure measurement datum, the received time datum, the received dispensed amount datum, and the received second time datum to monitor a condition of the eye.

### REFERENCE TO GOVERNMENT RIGHTS

This invention was made with United States government support awarded by the following agencies: National Institute of Health Grant No. R44 Ey014728-03. The United States government has certain rights in this invention.

### FIELD

The field of the disclosure relates generally to systems for monitoring characteristics of an eye.

### BACKGROUND

Glaucoma is a widespread disease that affects 1-2% of the population. An estimated 7 to 8 million Americans have an intraocular pressure (IOP) greater than 21 millimeters of mercury (mmHg) putting them at risk for optic nerve damage. Approximately 3.93 million Americans are diagnosed with glaucoma, and as a result, 900,000 have some degree of vision impairment, with 80,000 patients classified as legally blind. In the U.S., glaucoma is growing at a rate of 80,000 new cases annually. The economic loss resulting from vision impairment and blindness caused by glaucoma is estimated to be more than $1.5 billion per year.

The measurement of IOP (tonometry) requires an office visit with tests performed by a physician or trained technician. Current tonometry methods may not detect pressure peaks, and are known to both over- and under-estimate IOP. Even in patients diagnosed with persistent glaucoma, measurements of the intraocular pressure may be taken months apart. To properly manage glaucoma with medication, IOP measurements should be taken every few hours. Therefore, what is needed is a system for monitoring IOP regularly without a physician or clinician present.

### SUMMARY

In an example embodiment, a method for monitoring a condition of an eye is provided. An intraocular pressure measurement datum and a time datum associated with the time the intraocular pressure measurement was measured using an eye measurement system are received at a first device. A dispensed amount datum associated with an amount of a drug administered to an eye of a user and a second time datum associated with the time the drug was administered are received at the first device. The received intraocular pressure measurement datum, the received time datum, the received dispensed amount datum, and the received second time datum are stored at the first device to monitor a condition of the eye.

In an example embodiment, a system is provided to monitor a condition of an eye. The system includes, but is not limited to, a communication interface and a processor operably coupled to the communication interface. The communication interface is configured to receive an intraocular pressure measurement datum and a time datum associated with the time the intraocular pressure measurement was measured using an eye measurement system. The processor is configured to receive the intraocular pressure measurement datum and the time datum, to receive a dispensed amount datum associated with an amount of a drug administered to an eye of a user and a second time datum associated with the time the drug was administered, and to store the received intraocular pressure measurement datum, the received time datum, the received dispensed amount datum, and the received second time datum to monitor a condition of the eye.

In another example embodiment, a computer-readable medium is provided comprising computer-readable instructions that, upon execution by a processor, cause the processor to perform the operations of the method of monitoring a condition of an eye.

Other features and advantages of the invention will become apparent to those skilled in the art upon review of the following drawings, the detailed description, and the appended claims.

### BRIEF DESCRIPTION OF THE DRAWINGS

Example embodiments of the invention will hereafter be described with reference to the accompanying drawings, wherein like numerals denote like elements.

FIG. 1 depicts a block diagram of an eye monitoring system in accordance with an example embodiment.

FIG. 2 depicts a block diagram of a reader interfacing with an eye measurement system of the eye monitoring system of FIG. 1 in accordance with an example embodiment.

FIG. 3 depicts a block diagram of a reader of the eye monitoring system of FIG. 1 in accordance with an example embodiment.

FIG. 4 illustrates a front view of the reader of FIG. 3 in accordance with a first example embodiment.

FIG. 5 illustrates a front view of the reader of FIG. 3 in accordance with a first example embodiment.

FIG. 6 illustrates a front view of a dispenser of the eye monitoring system of FIG. 1 in accordance with a first example embodiment.

FIG. 7 illustrates a front view of the reader of FIG. 3 in accordance with a second example embodiment.

FIG. 8 illustrates a front view of the reader of FIG. 3 in accordance with a second example embodiment.

FIG. 9 depicts a flow diagram illustrating example operations performed by the eye monitoring system of FIG. 1 in accordance with an example embodiment.

FIG. 10 depicts a block diagram of the reader of FIG. 2 interfacing with a second eye measurement system of the eye monitoring system of FIG. 1 in accordance with a second example embodiment.

FIG. 11 depicts a flow diagram illustrating example operations performed by the eye monitoring system of FIG. 1 in accordance with a second example embodiment.

### DETAILED DESCRIPTION

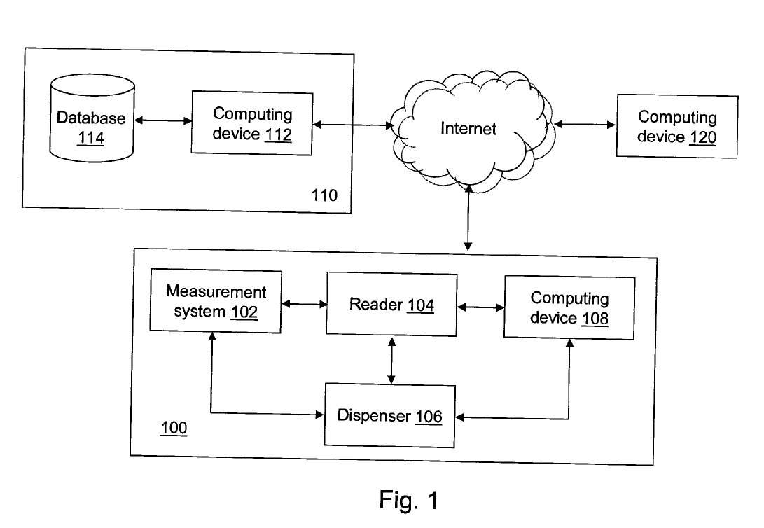

With reference to FIG. 1, a block diagram of an eye monitoring system 100 is shown in accordance with an example embodiment. Eye monitoring system 100 may include a measurement system 102, a reader 104, a dispenser 106, and a computing device 108. Measurement system 102 is placed on or in an eye of a patient to measure a chemical, biological, physical, and/or pharmacological level of a characteristic of the eye or of the patient. Measurement system 102 may also refer to devices that modify or regulate intraocular pressure. Measurement system 102 may include shunts, reservoirs, valves, drug eluting, and/or dispensing features. Measurement system 102 may be placed on an intraocular lens, an extraocular lens, a contact lens, a cataract lens, a pseudo-phakic lens, etc. without limitation. Intraocular implants are devices inserted in the eye. Intraocular placement areas for measurement system 102 include the anterior chamber, the posterior chamber, vitreous cavity, or on or within the sclera or other eye tissues. Measurement system 102 also may be integrated with a phakic or pseudophakic intraocular lens. Extraocular implants are devices outside of the sclera that may be placed in contact with the sclera or cornea. Devices placed in tissues near the eye are also considered extraocular. For example, a contact lens sensing device is an example of an extraocular device. Measurement system 102 also may include a combination of intraocular and extraocular components. For example, a sensor could be placed intraocular while supporting electronics are placed extraocular, for example, in the orbital space. The link between the sensor and supporting electronics may be wired or wireless.

Reader 104 receives the measured chemical, biological, physical, and/or pharmacological level from measurement system 102. Dispenser 106 dispenses medication such as a drug into the eye of the patient. Reader 104 and dispenser 106 may be integrated into a single device or included as separate devices. Computing device 108 may be a computer of any form factor including a laptop, a desktop, a server, an integrated messaging device, a personal digital assistant, a cellular telephone, an iPod, etc. Reader 104, dispenser 106, and computing device 108 may interact using a communication interface which may be wired or wireless or involve electrical connections in order to communicate information related to the condition of the eye of the patient or of the patient in general.

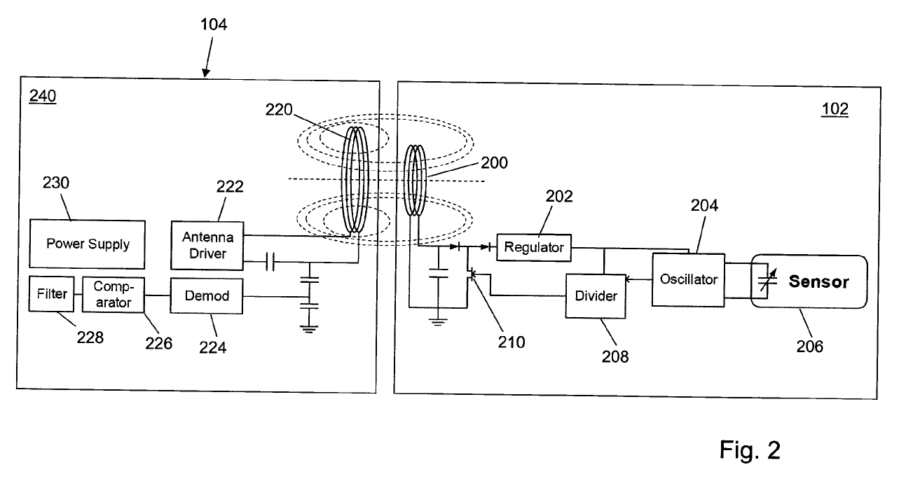

With reference to FIG. 2, a measurement reader 240 of reader 104 is shown interfacing with measurement system 102 to receive an intraocular pressure (IOP) measured by measurement system 102 in accordance with an example embodiment. In an example embodiment, measurement system 102 is surgically implanted in the vitreous chamber with the Kapton® substrate passing through the sclera. Measurement system 102 includes a custom integrated circuit (IC) and micro-electrical-mechanical system (MEMS) pressure sensor that may be mounted to an implanted intraocular lens to measure IOP. An intraocular lens may also be referred to as a phakic (measurement systemable contact lens) or a pseudo-phakic (cataract) intraocular lens. Additionally, the proposed method for measuring IOP may also be attached to a cataract lens or a pseudo-phakic lens.

Measurement system 102 includes, but is not limited to, a measurement system coil 200, a regulator 202, an oscillator 204, a sensor 206, a divider 208, and a MOSFET 210. Energy is supplied to the measurement system from a magnetic and/or an electric field produced in a reader coil 220 of reader 104. Measurement system coil 200 provides energy to regulator 202 which provides rectified and regulated power to oscillator 204 and to divider 208. Oscillator 204 determines an oscillating frequency based on a deflection of sensor 206. As known to those skilled in the art, a variety of antennas may be used instead of the coils indicated in the example embodiment of FIG. 2. In an example embodiment, sensor 206 is a capacitive pressure sensor. Divider 208 received the determined oscillating frequency, reduces the frequency, and drives MOSFET 210 that modulates the signal across measurement system coil 200. The modulated load is detected in reader coil 220.

In an example embodiment, sensor 206 is formed as a gap between parallel plates made by an etch into a surface of borosilicate glass. An electrode is patterned in the gap. Example materials for forming electrode are Ti/Pt, Cr/Au, Ti/Au, and Cr/Pt. However, almost any conducting material may be used. The electrode may be encased in a material that is biocompatible. The surface of a silicon wafer, 1-2 microns, is doped with boron using a thermal diffusion process to produce a thin, highly doped silicon layer that is resistant to wet etching by ethylenediamine pyrocatechol (EDP). After the boron diffusion, the wafer is aligned and anodically bonded to the patterned borosilicate glass. The glass/silicon assembly is placed in EDP to etch the entire silicon wafer away up to the p+ region until the remaining silicon p+ layer is about one micron thick and acts as a capacitive plate that deflects due to a pressure difference between the sealed cavity and the pressure of the external environment.

In an example embodiment, the components of measurement system 102 are mounted on a 51 micron thick Kapton® copper clad substrate. Connections between the components may be made using aluminum wire bonding. In an alternative embodiment, flip-chip bonding may be used to directly connect the components of measurement system 102 thereby eliminating most wire bonds. A one micron coating of parylene may be deposited on the components. Conformal epoxies may be overlaid to protect the wires from physical damage. A second deposition of parylene may be applied over measurement system 102.

In an example embodiment, measurement system coil 200 is formed by sputtering layers of titanium and gold onto a cured layer of polyimide. This layer is patterned using photolithography techniques and electroplated to a thickness of 10 microns. The remaining photoresist is removed and the non-electroplated gold seed layer is removed via wet etching methods. The exposed titanium layer is removed using dry plasma etching.

Measurement system 102 may include sensors of different types to measure IOP and may include sensors to measure different characteristics of the eye such as the glucose level, the temperature level, the pH level, etc. Additionally, in alternative embodiments, measurement system 102 may be powered by eye blinking, walking, solar energy, sound, light, vibration, and or a piezoelectric device. Measurement system 102 may be surgically implanted in one or more pieces. For example, measurement system 102 may include the measurement system integrated with a lens. Alternatively, the measurement system may be separate from the lens.

Measurement reader 240 of reader 104 includes reader coil 220, an antenna driver 222, a demodulator 224, a comparator 226, a filter 228, and a power supply 230. Antenna driver 222 provides the input signal to reader coil 220. Demodulator 224 receives an output signal of reader coil 220 which, in the example embodiment of FIG. 2, includes the IOP measured by sensor 206. Comparator 226 and filter 228 conditions the demodulated signal. The demodulation circuit extracts a data signal that has been modulated onto the carrier using amplitude-shift keying, phase-shift keying, differential phase-shift keying, frequency-shift keying, amplitude modulation, frequency modulation, pulsewidth modulation, or other standard modulation techniques used with radio-frequency identification devices. The comparator circuit converters the analog data signal to a digital data signal. The signal conditioner removes noise using analog, digital filters, or data filters. In an example embodiment, power supply 230 is a battery of any type.

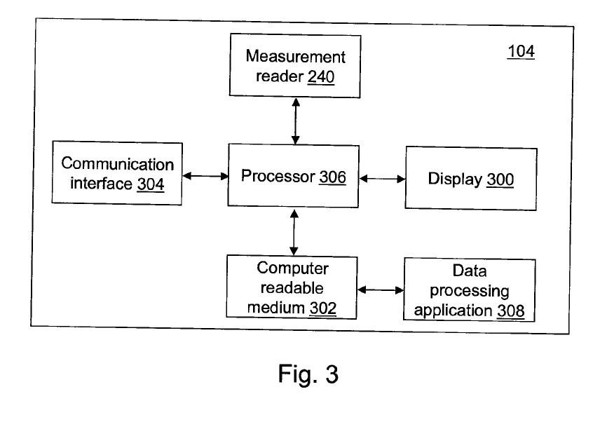

With reference to FIG. 3, reader 104 may further include a display 300, a computer-readable medium 302, a communication interface 304, a processor 306, and a data processing application 308. Different and additional components may be incorporated into reader 104. Display 300 presents information to a user of reader 104 as known to those skilled in the art. For example, display 300 may be a thin film transistor display, a light emitting diode (LED) display, a liquid crystal display, or any of a variety of different displays known to those skilled in the art now or in the future. In an example embodiment, display 300 presents the measured data to the user.

Computer-readable medium 302 is an electronic holding place or storage for information so that the information can be accessed by processor 306 as known to those skilled in the art. Computer-readable medium 302 can include, but is not limited to, any type of random access memory (RAM), any type of read only memory (ROM), any type of flash memory, etc. such as magnetic storage devices (e.g., hard disk, floppy disk, magnetic strips, . . . ), optical disks (e.g., compact disk (CD), digital versatile disk (DVD), . . . ), smart cards, flash memory devices, etc. Reader 104 may have one or more computer-readable media that use the same or a different memory media technology. Reader 104 also may have one or more drives that support the loading of a memory media such as a CD, a DVD, a flash memory card, etc.

Communication interface 304 provides an interface for receiving and transmitting data between devices using various protocols, transmission technologies, and media as known to those skilled in the art. The communication interface may support communication using various transmission media that may be wired or wireless. Example communication media, interfaces, and protocols include radio frequency wireless such as radio frequency identification (RFID), IEEE 802.11, IEEE 802.15, a cellular telephone network, etc.; a phone line; a power line; an infrared connection; a laser; an inductive coupling, a physical serial or parallel connection such as an Institute of Electrical and Electronics Engineers (IEEE) 1394 interface, an Ethernet interface, a universal serial bus interface, etc.

Processor 306 executes instructions as known to those skilled in the art. The instructions may be carried out by a special purpose computer, logic circuits, or hardware circuits. Thus, processor 306 may be implemented in hardware, firmware, software, or any combination of these methods. The term “execution” is the process of running an application or the carrying out of the operation called for by an instruction. The instructions may be written using one or more programming language, scripting language, assembly language, etc. Processor 306 executes an instruction, meaning that it performs the operations called for by that instruction. Processor 306 may operably couple with measurement reader 240, with display 300, with computer-readable medium 302, and with communication interface 304 to receive, to send, and to process information. Processor 306 may retrieve a set of instructions from a permanent memory device and copy the instructions in an executable form to a temporary memory device that is generally some form of RAM. Reader 104 may include a plurality of processors that use the same or a different processing technology.

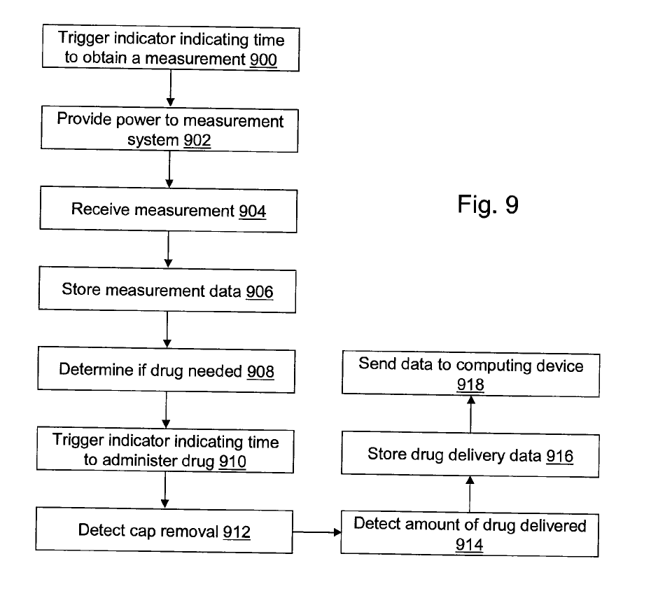

Data processing application 308 performs operations associated with storing and monitoring a measured characteristic of the eye and/or with indicating to a patient that administration of a medication is due. Some or all of the operations described with reference to FIG. 9 may be embodied in data processing application 308. The operations may be implemented using hardware, firmware, software, or any combination of these methods. With reference to the example embodiment of FIG. 3, data processing application 308 is implemented in software stored in computer-readable medium 302 and accessible by processor 306 for execution of the instructions that embody the operations of data processing application 308. Data processing application 308 may be written using one or more programming languages, assembly languages, scripting languages, etc.

Eye monitoring system 100 may interact with a server 110, for example, using communication interface 304. Server 110 may include a computing device 112 that can directly access or indirectly access a database 114. Communications between eye monitoring system 100 and server 110 may be established using secure communications through a network such as the Internet. The server may collect data from a plurality (thousands) of eye monitoring systems 100 such as an additional computing device similar to computing device 108 connected to the network.

Computing device 108 may submit IOP data (IOP and a timestamp) and dispenser data (amount of drug dispensed and a timestamp) with or without an associated patient identifier to server 110 automatically. For example, the IOP and dispenser data may be automatically sent to server 110 when a reading is obtained. The patient identifier may be associated with the wearer of measurement system 102, measurement system 102, reader 104, and/or dispenser 106. Alternatively, server 110 may interrogate each computing device 108 of the plurality of eye monitoring systems 100 for IOP data and dispenser data periodically. Server 110 may store the data received from multiple patients into database 114. The aggregated data in database 114 can be used for treatment analysis, drug effectiveness, etc. Physicians, ophthalmologists, and pharmacists can also review the data of their patients using a computing device 120 directly or indirectly connected to server 110 using a communication interface to a network such as the Internet as shown in FIG. 1.

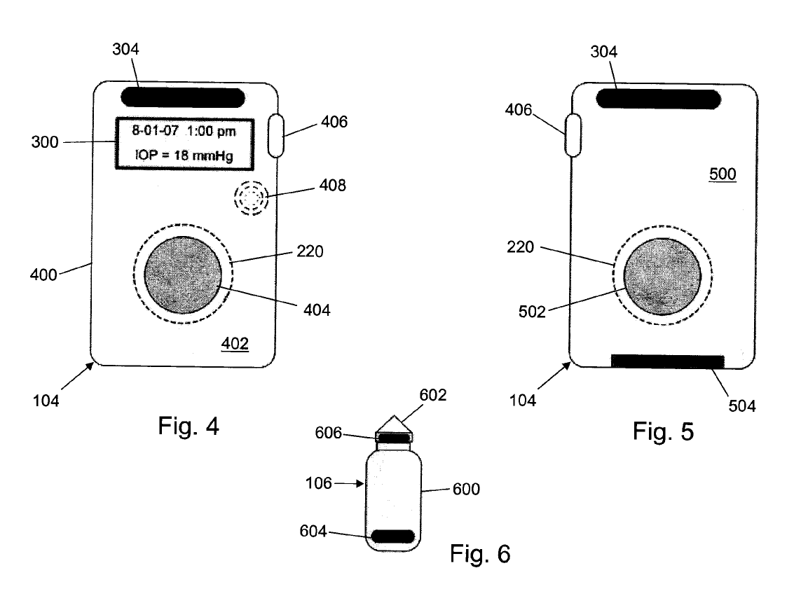

With reference to FIG. 4, a front view of reader 104 is shown in accordance with a first example embodiment. Reader 104 in accordance with the first example embodiment, includes display 300, reader coil 220, and communication interface 304 mounted in a body 400 that is generally rectangular similar to a hand held computing device such as a personal digital assistant, a cellular telephone, or an iPod. Other shapes may be used without limitation. Body 400 includes a front face 402 in which is mounted a front window 404. Front window 404 is mounted adjacent reader coil 220 to facilitate alignment of reader coil 220 with measurement system coil 200 when a sensor reading is performed. Display 300 is mounted in front face 402. As used in this disclosure, the term “mount” includes join, unite, connect, associate, insert, hang, hold, affix, attach, fasten, bind, paste, secure, bolt, screw, rivet, solder, weld, and other like terms. Thus, the elements of reader 104 can be mounted to or within body 400 using a variety of methods as known to those skilled in the art. Front face 402 further includes a button 406 and an indicator 408. Button 406 is a push button pushed by a user to perform a measurement using the sensor of measurement system 102 and reader 104. In the example embodiment of FIG. 4, indicator 408 is an LED that indicates that a sensor measurement should be performed by a user of reader 104. Other types of indicators may be used including sound, vibration, a message on display 300, etc. The indicated arrangement and mountings are merely example. Reader 104 may include additional components such as a second indicator to indicate that a drug should be delivered by a user of dispenser 106 and a clock to record timestamps.

With reference to FIG. 5, a back view of reader 104 is shown in accordance with the first example embodiment. Reader 104 includes a back face 500 in which is mounted a back window 502. Back window 502 is mounted adjacent reader coil 220 to facilitate alignment of reader coil 220 with measurement system coil 200 when a sensor reading is performed. Back face 500 further includes a drive 504 for a computer readable medium. In the example embodiment of FIG. 5, drive 504 is a flash memory card slot configured to accept a flash memory card. Data obtained by reader 104 may be stored on a computer readable medium inserted into drive 504 of reader 104. Additionally, reader 104 may utilize data stored on the computer readable medium to operate. For example, data processing application 308 may be stored on the computer readable medium. Reader 104 may further include a compartment for storing dispenser 106.

With reference to FIG. 6, dispenser 106 is shown in accordance with a first example embodiment. Dispenser 106 may be in the shape of a dropper bottle and include a body 600 and a cap 602. Dispenser 106 further includes a MEMS device for monitoring a drug delivery from dispenser 106 and communicating the amount of drug delivered to another device. For example, dispenser 106 may include a battery to charge a capacitive sensor 604 that senses the amount of drug delivered from dispenser 106, a clock, and a communication interface. In an example embodiment, the communication interface supports RFID. Dispenser 106 also may include a cap sensor 606 to sense that cap 602 has been removed or opened to deliver the drug stored in dispenser 106. Data on the amount of drug dispensed and a timestamp may be sent to reader 104 and/or computing device 108 using the communication interface. The indicated arrangement and mountings are merely example.

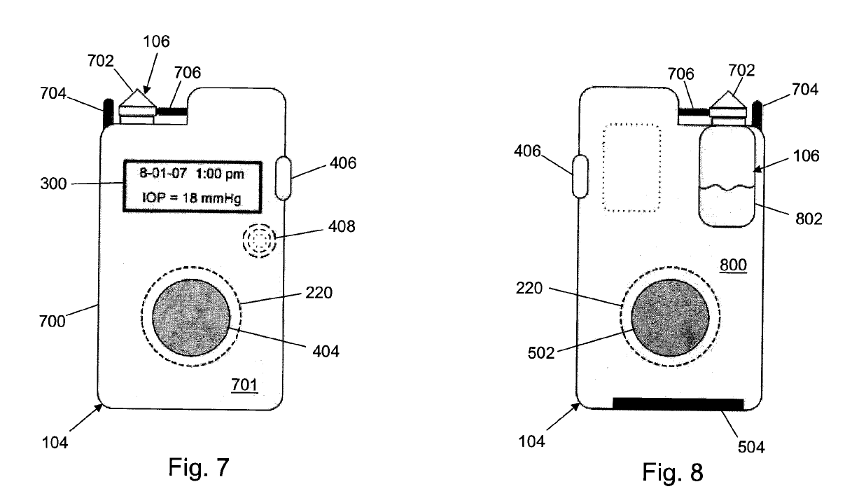

With reference to FIG. 7, a front view of reader 104 is shown in accordance with a second example embodiment including a dispenser 106 integrated with reader 104. Reader 104 in accordance with the second example embodiment, includes display 300 and reader coil 220 mounted in a body 700 that is generally rectangular similar to a hand held computing device such as a personal digital assistant, a cellular telephone, or an iPod. With reference to FIGS. 7 and 8, body 700 includes a front face 701, in which is mounted front window 404, display 300, indicator 408, and button 406, and a back face 800, in which is mounted back window 502. Back face 800 further includes drive 504 for a computer readable medium. The indicated arrangement and mountings are merely example.

Reader 104 in accordance with the second example embodiment, further includes dispenser 106 mounted in a compartment within body 700 in a manner facilitating dispensing of the drug stored in dispenser 106. With reference to FIGS. 7 and 8, dispenser 106 may be in the shape of a dropper bottle and include a body 802 and a cap 702. Reader 104, in accordance with the second example embodiment, further includes a drug delivery sensor 704 that senses the amount of drug delivered from dispenser 106 and a cap sensor 706 that senses that cap 702 has been removed or opened to deliver the drug stored in dispenser 106. Data on the amount of drug dispensed and a timestamp may be sent to reader 104 and/or computing device 108 using communication interface 304. The indicated arrangement and mountings are merely example.

Thus, dispenser 106 and reader 104 may be integrated into a single system. Dispenser 106, reader 104, and/or computing device 108 may be connected directly. For example, dispenser 106, reader 104, and/or computing device 108 may be connected using a cable for transmitting information between the devices. Dispenser 106, reader 104, and/or computing device 108 may be connected using a network using a wired or wireless media. Dispenser 106, reader 104, and/or computing device 108 may not be connected. Instead, data acquired using dispenser 106 and/or reader 104 may be manually provided to computing device 108. For example, the data may be stored on electronic media such as a CD, a DVD, a flash memory device, etc.

With reference to FIG. 9, example operations associated with eye monitoring system 100 and data processing application 308 are described. Additional, fewer, or different operations may be performed, depending on the embodiment. The order of presentation of the operations of FIG. 9 is not intended to be limiting. Additionally, the operations may be executed by a processor in one or more of dispenser 106 and/or of reader 104. In an example embodiment, the operations are executed in processor 306 of reader 104. In an operation 900, an indicator indicating time to obtain a sensor measurement is triggered. For example, the indicator may be indicator 406. In an example embodiment, the indicator may indicate a time to obtain an IOP reading. The indicator may be triggered periodically. Alternatively, the time to trigger the indicator may be calculated based on one or more of prior measured data, of a time of day, of an environmental condition such as a pressure or a temperature, of motion of the wearer of measurement system 102, etc. In another alternative embodiment, the indicator may be a phone call to the user.

In an operation 902, the user positions reader coil 220 in alignment with measurement system coil 200 and pushes button 406 which causes reader 104 to provide power to measurement system 102 through the field coupling the coils. In an operation 904, the sensor measurement performed by measurement system 102 is received at reader 104 through the field coupling the coils. In an operation 906, the received measurement datum is stored in computer readable medium 304. In an example embodiment, the measured datum is stored with a timestamp associated with the time the intraocular pressure measurement was measured using measurement system 102. The information may further be displayed to the user of reader 104 using display 300. The indicator may be turned “off” until triggered again, and parameters associated with triggering the indicator “on” may be reset.

In an operation 908, a determination of whether or not it is time to administer a drug is performed. If it is determined that it is time to administer a drug, in an operation 910, an indicator is triggered indicating that it is time to administer the drug to the eye of the user. In an operation 912, removal or opening of cap 602 is detected, for example using cap sensor 706. In an operation 914, an amount of the drug dispensed is detected, for example using drug delivery sensor 604, 704. In an operation 916, the drug delivery data is stored in computer readable medium 304 or at a computer readable medium of dispenser 106. For example, with reference to FIGS. 4-6, dispenser 106 detects the amount of drug dispensed using drug delivery sensor 604 and sends the detected amount to reader 104 which receives the amount of the drug dispensed using communication interface 304. With reference to FIG. 7 and 8, processor 306 receives the amount of the drug dispensed from drug delivery sensor 704 integrated with reader 104. In an example embodiment, the drug delivery data is stored with a timestamp associated with the time the drug was administered. The type of drug administered also may be stored. The information may further be displayed to the user of reader 104 using display 300. The indicator may be turned “off” until triggered again, and parameters associated with triggering the indicator “on” may be reset. In an operation 918, the received measurement data and/or drug delivery data is sent to computing device 108, for example using communication interface 304. Additional information also may be sent to computing device 108. For example, information identifying the user, information associated with the user, and/or information associated with measurement system 102 may further be sent to computing device 108 with or without a timestamp.

Reader 104 and/or dispenser 106 may be integrated into a variety of devices including, but not limited to, a watch, a key chain, a pager, a cell phone, a pair of glasses, another medical device such as a blood glucose monitor, a heart monitor, a medication container, etc. For example, reader 104 may be integrated into a pair of eye glasses that includes a battery such as a rechargeable lithium battery. Reader coil 220 may be mounted such that it is aligned with measurement system coil 200 when the glasses are worn. The glasses may be placed in a rechargeable cradle for recharging. An LED may be located on an inside frame of the glasses to alert the patient that an IOP threshold has been exceeded, and thus, to administer the drug. As another example, reader 104 or some components of reader 104 may be integrated into a headband or goggles. For example, the headband worn at night may send the measured data to the glasses that are being recharged. The headband also may be placed in the rechargeable cradle for recharging.

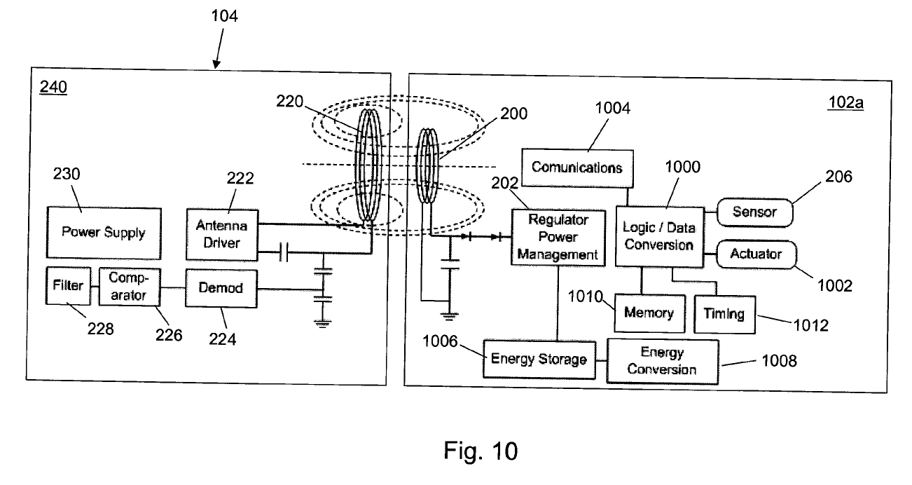

With reference to FIG. 10, a second measurement system 102a includes, but is not limited to, measurement system coil 200, sensor 206, power supply system 202, a logic and data conversion circuit 1000, an actuator interface 1002, and a communications interface 1004. Coil 200 may be used to receive energy from reader 104 for powering power supply system 202. Power supply system 202 may supply energy to a storage device 1006 such as a capacitor or a battery. The stored energy may be used to power sensor 206, logic and data conversion circuit 1000, actuator interface 1002, and communications interface 1004 and/or any other element functionally associated with second measurement system 102a. An energy conversion device 1008, such as a solar cell or energy harvesting structure, may also supply energy to storage device 1006. Logic and data conversion circuit 1000 measures the output of sensor 206 and may store the value in a memory 1010. The command to perform a measurement may initiate from reader 240 or from a timer circuit 1012 of second measurement system 102a. A command to operate actuator 1002 may occur the same way. Actuator control may also be based on sensor data. Communication to and from second measurement system 102a may occur through coil 200 or through communications interface 1004. Data sent to and received from second measurement system 102a may include values for, sensor measurements, configuration settings, device diagnostics, commands to perform functions, identification, calibration, error detection, etc. For example, data sent to and received from second measurement system 102a may include an eye measurement system identifier that identifies the specific measurement system 102, an eye measurement system type identifier that identifies the specific type of measurement system 102, a timestamp, an intraocular pressure measurement, a dispensed amount datum, a drug type identifier, a user identifier, a user age, a user gender, etc. any or all of the data items may be sent between multiple devices and types of devices.

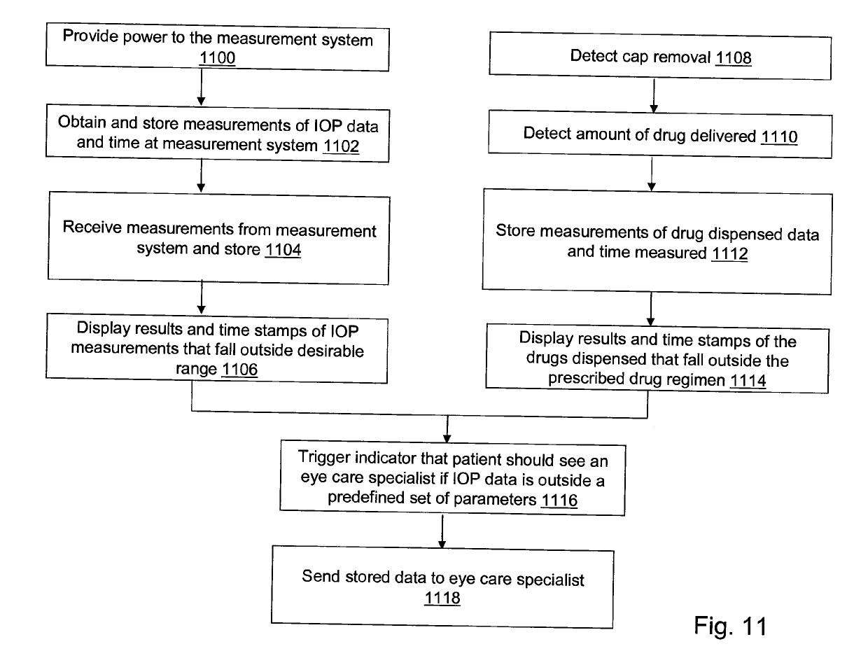

With reference to FIG. 11, example operations associated with eye monitoring system 100 and data processing application 308 are described. Additional, fewer, or different operations may be performed, depending on the embodiment. The order of presentation of the operations of FIG. 11 is not intended to be limiting. Additionally, the operations may be executed by a processor in one or more of dispenser 106, of reader 104, of computing device 108, and/or of second measurement system 102a. In an operation 1100, power is provided to measurement system 102a. In operation 1102, an IOP measurement and time are obtained and stored at measurement system 102a. In an operation 1104, the IOP measurement and time are received from measurement system 102a and stored. For example, the data may be received and stored at one or more of dispenser 106, reader 104, and computing device 108 so that the data can be evaluated by an eye care specialist during a future office visit. In an operation 1106, the IOP measurement and time that fall outside a desirable range may be displayed to the user, for example, using one or more of dispenser 106, reader 104, and computing device 108. Processing continues at an operation 1116.

In a separate process that may be executing concurrently with operations 1100, 1102, 1104, and 1106, in an operation 1108, a cap removal from dispenser 106 is detected. In an operation 1110, an amount of drug delivered from dispenser 106 is detected. In another example embodiment, the amount of drug may not be detected, but may be assumed to be a predefined amount that is triggered based on detection of the cap removal. Thus, a dispensed amount may be received into the processor that is based on a detected amount of drug delivered from dispenser 106 or based on a predefined amount that is stored in a computer-readable medium. In an operation 1112, the dispensed drug data and time are stored. For example, the data may be received and stored at one or more of dispenser 106, reader 104, and computing device 108 so that the data can be evaluated by an eye care specialist during a future office visit. In an operation 1114, the dispensed drug data and time that fall outside a prescribed drug regimen may be displayed to the user, for example, using one or more of dispenser 106, reader 104, and computing device 108. Processing continues at operation 1116.

In operation 1116, an indicator indicating that the user should see an eye care specialist is triggered. For example, if the IOP data or data associated with the IOP data such as minimum IOP, maximum IOP, and/or rate of change of IOP are outside a predefined set of parameters, the indicator may be triggered. In an operation 1118, the stored data is sent to the eye care specialist using a communication interface from one or more of dispenser 106, reader 104, and computing device 108.

In an operation 902, the user positions reader coil 220 in alignment with measurement system coil 200 and pushes button 406 which causes reader 104 to provide power to measurement system 102 through the field coupling the coils. In an operation 904, the sensor measurement performed by measurement system 102 is received at reader 104 through the field coupling the coils. In an operation 906, the received measurement datum is stored in computer readable medium 304. In an example embodiment, the measured datum is stored with a timestamp associated with the time the intraocular pressure measurement was measured using measurement system 102. The information may further be displayed to the user of reader 104 using display 300. The indicator may be turned “off” until triggered again, and parameters associated with triggering the indicator “on” may be reset.

In an operation 908, a determination of whether or not it is time to administer a drug is performed. If it is determined that it is time to administer a drug, in an operation 910, an indicator is triggered indicating that it is time to administer the drug to the eye of the user. In an operation 912, removal or opening of cap 602 is detected, for example using cap sensor 706. In an operation 914, an amount of the drug dispensed is detected, for example using drug delivery sensor 604, 704. In an operation 916, the drug delivery data is stored in computer readable medium 304 or at a computer readable medium of dispenser 106. For example, with reference to FIGS. 4-6, dispenser 106 detects the amount of drug dispensed using drug delivery sensor 604 and sends the detected amount to reader 104 which receives the amount of the drug dispensed using communication interface 304. With reference to FIG. 7 and 8, processor 306 receives the amount of the drug dispensed from drug delivery sensor 704 integrated with reader 104. In an example embodiment, the drug delivery data is stored with a timestamp associated with the time the drug was administered. The type of drug administered also may be stored. The information may further be displayed to the user of reader 104 using display 300. The indicator may be turned “off” until triggered again, and parameters associated with triggering the indicator “on” may be reset. In an operation 918, the received measurement data and/or drug delivery data is sent to computing device 108, for example using communication interface 304. Additional information also may be sent to computing device 108. For example, information identifying the user, information associated with the user, and/or information associated with measurement system 102 may further be sent to computing device 108 with or without a timestamp.

In an alternative embodiment, a “passive” telemetry device may be used that is simpler on the measurement system side because it only contains a resonant inductor and capacitor (LC) circuit. However, a “passive” telemetry device is generally more expensive and bulky on the reader side. In an example embodiment using a “passive” telemetry device, measurement system 102 may be constructed of passive components where the resonant frequency, phase, or some characteristic of the electromagnetic response of the measurement system is a function of pressure. Passive measurement systems may include inductors, capacitors, capacitive or inductive sensors, surface acoustic wave devices, crystals, resonating MEMS structures, and antennas. Semiconductors may also be included as safety devices or elements to shape the electromagnetic response. The measurement system reader may interrogate the passive measurement system using a swept frequency electromagnetic field so that the measurement system resonates and produces a detectable signal. The measurement system reader may also generate a pulsed electromagnetic field and wirelessly sense the resonating measurement system. Any portion of the electromagnetic spectrum may be used including visible and invisible light.

The word “example” is used herein to mean serving as an example, instance, or illustration. Any aspect or design described herein as “example” is not necessarily to be construed as preferred or advantageous over other aspects or designs. Further, for the purposes of this disclosure and unless otherwise specified, “a” or “an” means “one or more”. The example embodiments may be implemented as a method, machine, or article of manufacture using standard programming and/or engineering techniques to produce software, firmware, hardware, or any combination thereof to control a computer to implement the disclosed embodiments.

The foregoing description of example embodiments of the invention have been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A system, the system comprising:
a communication interface configured to receive an intraocular pressure measurement datum and a time datum associated with the time the intraocular pressure measurement was measured using an eye measurement system; and
a processor operably coupled to the communication interface to receive the intraocular pressure measurement datum and the time datum, the processor configured to
receive a dispensed amount datum associated with an amount of a drug administered to an eye of a user and a second time datum associated with the time the drug was administered; and
store the received intraocular pressure measurement datum, the received time datum, the received dispensed amount datum, and the received second time datum to monitor a condition of the eye.

2. The system of claim 1, wherein the communication interface is further configured to receive the dispensed amount datum.

3. The system of claim 1, further comprising a drug dispenser and a sensor configured to detect the amount of the drug dispensed to the eye of the user using the drug dispenser.

4. The system of claim 1, further comprising a display to present the received intraocular pressure measurement datum to the user.

5. The system of claim 1, further comprising a display to present the received dispensed amount datum to the user.

6. The system of claim 1, wherein the communication interface is further configured to send the received intraocular pressure measurement datum and the received dispensed amount datum to a second device.

7. The system of claim 1, wherein the communication interface comprises an antenna configured to generate a field that couples with a second antenna of the eye measurement system to receive the intraocular pressure measurement datum.

8. The system of claim 7, wherein the antenna is a coil antenna.

9. The system of claim 7, wherein the field that couples with the second antenna of the eye measurement system provides energy to the eye measurement system.

10. The system of claim 7, further comprising the eye measurement system, wherein the eye measurement system comprises the second antenna and a sensor operably coupled to the second antenna and configured to obtain the intraocular pressure measurement.

11. The system of claim 1, wherein the eye measurement system is mounted on an intraocular lens.

12. The system of claim 1, wherein the eye measurement system is mounted within an intraocular lens.

13. The system of claim 1, further comprising an indicator configured to indicate that administration of the drug is needed.

14. The system of claim 1, wherein the processor is further configured to determine if an office visit to an eye care specialist is needed based on data associated with the received intraocular pressure measurement.

15. The system of claim 1, further comprising an indicator configured to indicate that the intraocular pressure measurement is needed.

16. A computer-readable medium comprising computer-readable instructions therein that, upon execution by a processor, cause the processor to monitor a condition of an eye, the instructions configured to cause a computing device to:
receive an intraocular pressure measurement datum and a time datum associated with the time the intraocular pressure measurement was measured using an eye measurement system;
receive a dispensed amount datum associated with an amount of a drug administered to an eye of a user and a second time datum associated with the time the drug was administered; and
store the received intraocular pressure measurement datum, the received time datum, the received dispensed amount datum, and the received second time datum to monitor a condition of the eye.

17. A method of monitoring a condition of an eye, the method comprising:
receiving an intraocular pressure measurement datum and a time datum associated with the time the intraocular pressure measurement was measured using an eye measurement system at a first device;
receiving a dispensed amount datum associated with an amount of a drug administered to an eye of a user and a second time datum associated with the time the drug was administered at the first device; and
storing the received intraocular pressure measurement datum, the received time datum, the received dispensed amount datum, and the received second time datum at the first device to monitor a condition of the eye.

18. The method of claim 17, further comprising sending the stored intraocular pressure measurement datum, the stored time datum, the stored dispensed amount datum, and the stored second time datum from the first device to a second device.

19. The method of claim 17, further comprising detecting, at the first device, the amount of the drug administered to the eye of a user.

20. The method of claim 17, further comprising detecting, at the first device, a cap removal from a dispenser used to administer the drug to the eye of a user.

21. The method of claim 17, further comprising providing power to the eye measurement system from the first device while receiving the measurement.

22. The method of claim 17, further comprising triggering an indicator to indicate that administration of the drug is needed.

23. The method of claim 17, further comprising determining if administration of the drug is needed based on the received intraocular pressure measurement.

24. The method of claim 17, further comprising triggering an indicator to indicate that the intraocular pressure measurement is needed.

25. The method of claim 24, further comprising determining when to trigger the indicator before triggering the indicator.

26. The method of claim 25, wherein the determination is based on one or more parameter selected from the group consisting of one or more received measurement, a time of day, a pressure measurement, a temperature measurement, and a movement of the user.
